(19) United States
(12) Patent Application Publication
(10) Pub. No.: US 2010/0086483 A1
(43) Pub. Date: Apr. 8, 2010

(54) METHOD OF MULTIDETECTOR COMPUTED TOMAGRAPHY

(75) Inventors: Luiz Belardinelli, Palo Alto, CA (US); Brent Blackburn, Los Altos, CA (US)

Correspondence Address: Swiss Tanner, P.C.;P.O. Box 1749, Four Main Street, Suite 100, Los Altos, CA, 94022, US

(73) Assignee: Gilead Palo Alto, Inc.

(21) Appl. No.: 12/569,643

(22) Filed: Sep. 29, 2009

Related U.S. Application Data
(60) Provisional application No. 61/101,043, filed on Sep. 29, 2008.

(51) Int. Cl. A61K 49/00; A61K 31/7076; A61K 31/522

(52) U.S. Cl. 424/9.1; 514/46; 514/263.34

(57) ABSTRACT

This invention relates to methods for multidetector computed tomography myocardial perfusion imaging comprising administering doses of a rate-control agent and one or more adenosine A_{2A} receptor agonists to a mammal.

### CROSS REFERENCE TO RELATED APPLICATIONS

This application claims the benefit under 35 U.S.C. §119(e) of U.S. Provisional Application 61/101,043 filed on Sep. 29, 2008, which is hereby incorporated by reference in its entirety.

### FIELD OF THE INVENTION

This invention relates to methods for multidetector computed tomography myocardial perfusion imaging comprising administering doses of a rate-control agent and one or more adenosine A_{2A} receptor agonists to a mammal.

### BACKGROUND OF THE INVENTION

In recent years, multidetector computed tomography (MDCT) has been used in the diagnosis of coronary artery diseases, Kido et al. (2008) Circ J, 72:1086-1091 and George et al. (2206) JACC 48(1):153-160. Advantages for using MDCT are more accuracy, less radiation exposure and shorter scan time (20 to 30 seconds). However, it requires a lower heart rate to increase the cardiac rest period and to reduce the motion artifacts. In MDCT, β-adrenergic blockers have previously been used to reduce the heart rate. Unfortunately, the use of β-adrenergic blockers is also known to increase myocardial blood flow.

Regadenoson (CVT-3146) is an A_{2A} adenosine receptor agonist and was approved by the US FDA in 2008 for use as a coronary vasodilator in pharmacologic stress testing for myocardial perfusion imaging. Regadenoson is a selective and potent coronary vasodilator which, unlike adenosine, may be administered in a weight-independent bolus dose. The use of adenosine is limited due to side effects such as flushing, chest discomfort, the urge to breathe deeply, headache, throat, neck, and jaw pain. These adverse effects of adenosine are due to the activation of other adenosine receptor subtypes in addition to A_{2A}, which mediates the vasodilatory effects of adenosine. Additionally, the short half-life of adenosine necessitates multiple treatments during the procedure, further limiting its use.

The ability of regadenoson to be administered as a bolus dose makes it an extremely attractive agent for us in MDCT. The suitability of regadenoson for use in MDCT is however complicated by the fact that it is also causes an increase in heart rate. Thus, there is still a need for a method of eliminating the increase in heart rate associated with the administration of regadenoson, which would be useful for myocardial perfusion imaging with MDCT. Preferred compounds would be selective for the A_{2A} adenosine receptor and have a short duration of action (although longer acting than compounds such as adenosine), thus obviating the need for multiple dosing.

### SUMMARY OF THE INVENTION

This invention is directed to the surprising discovery that an A_{2A} adenosine receptor agonist, when administered to a patient together with a rate control agent, such as β adrenergic blocker and/or caffeine, can be used in conjunction with multidetector computed tomography to diagnose coronary disease in the patient.

The following are aspects of this invention:

A pharmaceutical composition comprising a rate control agent, at least 10 μg of at least one A_{2A} receptor agonist, and at least one pharmaceutically acceptable carrier.

A method of vasodilator induced myocardial stress perfusion multidetector computed tomography imaging of a myocardium of a mammal, comprising administering a therapeutically effective amount of a rate control agent and at least 10 μg of at least one A_{2A} receptor agonist to the mammal and imaging the myocardium of the mammal.

A method of vasodilator induced myocardial stress perfusion multidetector computed tomography imaging of a myocardium of a mammal, comprising administering a therapeutically effective amount of a rate control agent and no more than about 1000 μg of at least one A_{2A} receptor agonist to the mammal and imaging the myocardium of the mammal.

A method of vasodilator induced myocardial stress perfusion multidetector computed tomography imaging of a mammal, comprising administering a therapeutically effective amount of a rate control agent and at least 10 μg of at least one A_{2A} receptor agonist to the mammal wherein the rate control agent is administered to the mammal before or concurrently with the at least one A_{2A} receptor agonist.

A method of vasodilator induced myocardial stress perfusion multidetector computed tomography imaging of a mammal, comprising administering a rate control agent and no more than about 1000 μg of an A_{2A} receptor agonist to the mammal.

A method of vasodilator induced myocardial stress perfusion multidetector computed tomography imaging of a mammal, comprising administering a rate control agent and an A_{2A} receptor agonist in an amount ranging from about 10 to about 600 μg to the mammal.

A method of vasodilator induced myocardial stress perfusion multidetector computed tomography imaging of a mammal, comprising administering a rate control agent and an A_{2A} receptor agonist in an amount ranging from about 10 to about 600 μg to the mammal, wherein the A_{2A} receptor agonist is administered in less than about 10 seconds.

A method of vasodilator induced myocardial stress perfusion multidetector computed tomography imaging of a mammal, comprising administering a rate control agent and an A_{2A} receptor agonist in an amount ranging from about 10 to about 600 μg to the mammal, wherein the A_{2A} receptor agonist is administered in an amount greater than about 10 μg.

A method of vasodilator induced myocardial stress perfusion multidetector computed tomography imaging of a mammal, comprising administering a rate control agent and an A_{2A} receptor agonist in an amount ranging from about 10 to about 600 μg to the mammal, wherein the A_{2A} receptor agonist is administered in an amount greater than about 100 μg.

A method of vasodilator induced myocardial stress perfusion multidetector computed tomography imaging of a mammal, comprising administering a rate control agent and an A_{2A} receptor agonist in an amount ranging from about 10 to about 600 μg to the mammal, wherein the A_{2A} receptor agonist is administered in an amount no greater than 600 μg.

A method of vasodilator induced myocardial stress perfusion multidetector computed tomography imaging of a mammal, comprising administering a rate control agent and an A_{2A} receptor agonist in an amount ranging from about 10 to about 600 μg to the mammal, wherein the A_{2A} receptor agonist is administered in an amount no greater than 500 μg.

A method of vasodilator induced myocardial stress perfusion multidetector computed tomography imaging of a mammal, comprising administering a rate control agent and an A_{2A} receptor agonist in an amount ranging from about 10 to about 600 μg to the mammal, wherein the A_{2A} receptor agonist is administered in an amount ranging from about 100 μg to about 500 μg.

A method of vasodilator induced myocardial stress perfusion multidetector computed tomography imaging of a mammal, comprising administering a rate control agent and an A_{2A} receptor agonist in an amount ranging from about 10 to about 600 μg to the mammal, wherein the A_{2A} receptor agonist is selected from the group consisting of CVT-3033, regadenoson, and combinations thereof.

A method of vasodilator induced myocardial stress perfusion multidetector computed tomography imaging of a mammal, comprising administering a rate control agent and regadenoson in an amount ranging from about 10 to about 600 μg in a single IV bolus.

A method of vasodilator induced myocardial stress perfusion multidetector computed tomography imaging of a mammal, comprising administering a rate control agent and regadenoson in an amount ranging from about 100 to about 500 μg in a single IV bolus.

In all of the methods above, the mammal is typically a human.

In all of the methods above, the dose is typically administered in a single IV bolus.

In all of the method above, the rate control agent may be any agent capable of reducing the increase in heart rate associated with the administration of an A_{2A} agonist. Suitable rate control agents include but are not limited to caffeine and other non-selective adenosine antagonists such as, for example, aminophylline caffeine, dyphylline, enprophylline, pentoxyphylline, and theophylline and β-adrenergic receptor blocker such metoprolol and propranolol.

### DETAILED DESCRIPTION OF THE INVENTION

### Definitions and General Parameters

Unless defined otherwise, all technical, and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

It must be noted that as used herein, and in the appended claims, the singular forms “a,” “an,” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification methods of the components of the compositions disclosed herein. “Consisting of” shall mean excluding more than trace elements of other ingredients of the compositions of this invention. Embodiments defined by each of these transition terms are within the scope of this invention.

“Optional” or “optionally” means that the subsequently described event or circumstance may or may not occur, and that the description includes instances where said event or circumstance occurs and instances in which it does not.

The term “beta-blocker” refers to an agent that binds to a beta-adrenergic receptor and inhibits the effects of beta-adrenergic stimulation. Beta-blockers increase AV nodal conduction. In addition, Beta-blockers decrease heart rate by blocking the effect of norepinephrine on the post synaptic nerve terminal that controls heart rate. Beta blockers also decrease intracellular Ca++ overload, which inhibits after-depolarization mediated automaticity. Examples of beta-blockers include, but are not limited to, acebutolol, albuterol, amosulalol, arotinolol, atenolol, befunolol, betaxolol, bevantolol, bisoprolol, bisoprolol fumarate, bopindolol, bucindolol, bufetolol, bunitrolol, butaxamine, butofilolol, carazolol, carteolol, carvedilol, celiprolol, cloranolol, divalproex, epanolol, carvedilol, esmolol, indenolol, landiolol, labetalol, levobunolol, levomoprolol, lisinopril, medroxalol, mepindolol, metipranolol, metoprolol, nadolol, nebivolol, nifenalol, nipradilol, oxprenolol, penbutolol, pindolol, propafenone, propranolol, salmeterol, sotalol, talinolol, tertatolol, tilisolol, timolol, verapamil, xamoterol, and xibenolol.

The term “therapeutically effective amount” refers to that amount of a rate control agent that is sufficient to effect treatment, as defined below, when administered to a mammal in need of such treatment. In other words, this term could also be referred to as the heart-rate controlling amount when the rate control agent is administered in combination with the A_{2A} receptor agonist to provide for conditions sufficient to image the myocardium of the patient. The therapeutically effective amount will vary depending upon the specific activity of the therapeutic agent being used, the severity of the patient's disease state, and the age, physical condition, existence of other disease states, and nutritional status of the patient. Additionally, other medication the patient may be receiving will effect the determination of the therapeutically effective amount of the therapeutic agent to administer.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

As used herein “multidetector computed tomography” or “MDCT” may also be referred to as multidetector CT, multidetector-row computed tomography, multidetector-row CT, multisection CT, multislice computed tomography, and multislice CT.

### EMBODIMENTS OF THE INVENTION

New and potent partial A_{2A} agonists that increase CBF but do not significantly increase peripheral blood flow have been identified. The partial A_{2A} agonists, including regadenoson and CVT-3033 have a rapid onset and a short duration when administered. An unexpected and newly identified benefit of these new compounds is that they are very useful when administered in a very small quantity in a single bolus intravenous injection. The partial A_{2A} receptor agonists can be administered in amounts as little as 10 μg and as high as 600 μg or more and still be effective with few if any side-effects. An optimal intravenous dose will include from about 100 to about 500 μg of at least one partial A_{2A} receptor agonist. This amount is unexpectedly small when compared with adenosine which is typically administered in continuously by IV at a rate of about 140 μg/kg/min. Unlike adenosine, the same dosage of partial A_{2A} receptor agonists, and in particular, regadenoson and CVT-3033 can be administered to a human patient regardless of the patient's weight. Thus, the administration of a single uniform amount of an A_{2A} receptor agonists by IV bolus for myocardial imaging is dramatically simpler and less error prone than the time and weight dependent administration of adenosine.

Other selective agonists for the A_{2A} adenosine receptor are also known and are also suitable for use in the methods of the invention. For example, MRE-0470 (Medco) is an adenosine A_{2A} receptor agonist that is a potent and selective derivative of adenosine which may be used as an adjuvant in imaging. MRE-0470, also known as binodenoson, is typically administered by IV bolus or IV infusion with a typical dose being 1.5 mcg/kg bolus or 1.5 mcg/kg/min. See Udelson et al., Circulation. 2004 Feb. 3; 109(4):457-64.

It has been discovered that surprisingly when administered with a suitable rate control agent, A_{2A} receptor agonists may also be used in perfusion MDCT myocardial imaging. MDCT is a form of computed tomography (CT) technology for diagnostic imaging. In MDCT, a two-dimensional array of detector elements replaces the linear array of detector elements used in typical conventional and helical CT scanners. The two-dimensional detector array permits CT scanners to acquire multiple slices or sections simultaneously and greatly increase the speed of CT image acquisition. Image reconstruction in MDCT is more complicated than that in single section CT. Nonetheless, the development of MDCT has resulted in the development of high resolution CT applications such as CT angiography and CT colonoscopy (see, MHK Hoffmann, et al. American Journal of Roentgenology, 2004, 182:601-608).

The rate control agent can be administered to the patient prior to administration of an A_{2A} receptor agonist. Prior administration refers to administration at a time before administration of the A_{2A} receptor agonist that allows a therapeutically effective amount of the rate control agent to remain in the mammal's blood at the time of the administration of the A_{2A} receptor agonist. More preferably, prior administration refers to administration of caffeine no greater than about 120 minutes before and even more preferably no greater than 30 minutes before administration of the A_{2A} receptor agonist.

Alternatively, the rate control agent can be administered at the same time as the A_{2A} receptor agonist. Towards this end, the rate control agent can be incorporated into the A_{2A} receptor agonist containing pharmaceutical composition or it can be administered as a separate pharmaceutical composition.

The rate control agent will be administered to mammals according to the methods and compositions of this invention in a therapeutically effective amount. The therapeutically effective amount will be an amount of caffeine that is sufficient to provide for a heart rate below 100 beats per minute. When the non-selective adenosine receptor antagonist caffeine is used for example, the therapeutically effective amount will be a dose of caffeine ranging from about 50 mg to about 1000 mg. More preferably, the dose of caffeine will range from about 100 mg to about 500 mg. Most preferably, the dose of caffeine will range from about 200 mg to about 400 mg.

The compositions may be administered orally, intravenously, through the epidermis or by any other means known in the art for administering therapeutic agents with bolus IV administration being preferred.

The rate control agent may be administered to the mammal in a liquid or solid pharmaceutical dosage. As discussed above, the rate control agent may be administered with or independently from the A_{2A} receptor agonist. If the rate control agent is administered with the A_{2A} receptor agonist, then it is preferred that the combination is administered as a single IV bolus. If the rate control agent is administered independently. i.e., separately from the A_{2A} receptor agonist, then the rate control agent can be administered in any known manner including by way of a solid oral dosage form such as a tablet or by way of an IV infusion or IV bolus.

Pharmaceutical compositions including the compounds of this invention, and/or derivatives thereof, may be formulated as solutions or lyophilized powders for parenteral administration. Powders may be reconstituted by addition of a suitable diluent or other pharmaceutically acceptable carrier prior to use. If used in liquid form the compositions of this invention are preferably incorporated into a buffered, isotonic, aqueous solution. Examples of suitable diluents are normal isotonic saline solution, standard 5% dextrose in water and buffered sodium or ammonium acetate solution. Such liquid formulations are suitable for parenteral administration, but may also be used for oral administration. It may be desirable to add excipients such as polyvinylpyrrolidinone, gelatin, hydroxy cellulose, acacia, polyethylene glycol, mannitol, sodium chloride, sodium citrate or any other excipient known to one of skill in the art to pharmaceutical compositions including compounds of this invention.

A very useful and potent and selective agonists for the A_{2A} adenosine receptor is regadenoson or (1-{9-[(4S,2R,3R,5R)-3,4-dihydroxy-5-(hydroxymethyl)oxolan-2-yl]-6-aminopurin-2-yl}pyrazol-4-yl)-N-methylcarboxamide which has the formula:

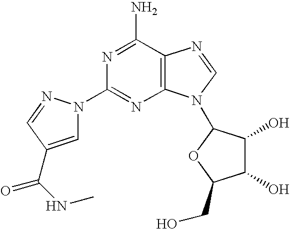

Another preferred compound that is useful as a selective A_{2A}-adenosine receptor agonist with a short duration of action is a compound of the formula:

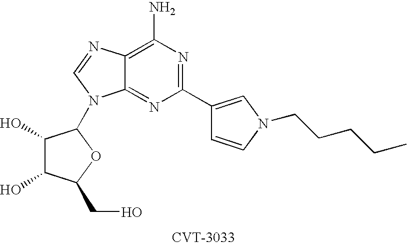

CVT-3033, having the chemical name (3S,4R,5S)-2-(6-amino-2-(1-pentyl-1H-pyrazol-4-yl)-9H-purin-9-yl)-5-(hydroxymethyl)tetrahydrofuran-3,4-diol, is particularly useful as an adjuvant in cardiological imaging.

Other of compounds that are suitable for use in the method of the invention are described in more detail in U.S. Pat. Nos. 6,403,567 and 6,214,807, the specification of each of which is incorporated herein by reference. Additional classes of compounds that are suitable for use in the methods of the invention are also identified and discussed in detail in U.S. Pat. Nos. 5,278,150, 6,322,771, and 7,214,665 as well as PCT Publications WO 2006/076698 and WO 1999/034804.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Any methods that are functionally equivalent are within the scope of the invention. Various modifications of the invention in addition to those described herein will become apparent to those skilled in the art from the foregoing description and accompanying figures. Such modifications fall within the scope of the appended claims.

Unless otherwise stated all temperatures are in degrees Celsius. Also, in these examples and elsewhere, abbreviations have the following meanings:
μg=microgram
μM=micromolar
AE=adverse event
AV=atrioventricular
bpm=beats per minute
CBF=coronary blood flow
ECG=electrocardiogram
HR=heart rate
IM=intramuscularly
IV=intravenous
kg=kilogram
LV dP/dt_{Max}=Maximum rate of rise of left ventricular pressure
LVSP=left ventricular systolic pressure
MAP=mean arterial pressure
mg=milligram
min=minute
mL=milliliter
mm=millimeter
msec=millisecond
NS=not significant
PO or po=oral
sec=second
SEM=standard error of the mean

### Example 1

### Background

Regadenoson (Reg), an A_{2A} adenosine receptor agonist and coronary vasodilator, is approved as a pharmacologic stress agent for myocardial perfusion imaging. Reg can cause sympathoexcitation and tachycardia. In recent years, multi-detector computed tomography (MDCT) has been used in the diagnosis of coronary artery diseases. Advantages for using MDCT are more accuracy, less radiation exposure and shorter scan time (20 to 30 sec). However, it requires a lower heart rate to increase the cardiac rest period and to reduce motion artifacts. In MDCT, β-adrenergic blockers may be used to reduce the heart rate (HR). Our goal was to determine whether β₁-adrenergic blockade can inhibit tachycardia without decreasing coronary vasodilation induced by Reg in conscious dogs.

### Methods:

Five mongrel dogs were chronically instrumented for measurements of systemic hemodynamics and coronary blood flow (CBF). The effects of regadenoson (1, 2.5 and 5 μg/kg, IV) on HR and CBF were assessed before and after administration of the β₁-adrenergic receptor blocker metoprolol (1.5 mg/kg). Values of peak CBF and the duration of the two-fold increase in CBF above baseline were used to assess Reg-induced coronary vasodilation.

### Results:

Reg (1, 2.5 and 5 μg/kg) caused a dose-dependent increase in peak CBF (ΔCBF: 129±10, 149±7 and 174±10 mL/min, respectively, mean±SEM, n=4-5, all p<0.05) and in duration of hyperemia. The durations of 2-fold increases in CBF were 93±22, 316±57 and 593±86 sec at 1, 2.5 and 5 μg/kg Reg, respectively. Reg also caused a dose-dependent increase in HR (ΔHR: 49±8, 63±5, and 71±7 bpm, respectively, all p<0.05). The Reg-induced tachycardia was markedly reduced after IV administration of metoprolol (ΔHR: 19±4, 28±3, and 39±5 bpm at 1, 2.5 and 5 μg/kg Reg, respectively, all p<0.05 versus control) to 55±12, 54±7 and 45±4% of control. The Reg (1, 2.5 and 5 μg/kg)-induced coronary vasodilation was reduced in the presence of metoprolol by 11±7, 10±4 and 21±2% from control (ΔCBF: 112±5 (NS), 136±16 (NS) and 138±9 (p<0.05) mL/min, respectively) and the durations of two-fold increases in CBF were reduced to 71±34, 215±45 and 364±86 sec, respectively (p<0.05 versus control).

### Conclusion:

Our results indicate that 1-5 μg/kg regadenoson caused a dose-dependent coronary vasodilation and an increase in HR. β₁-Adrenergic blockade with metoprolol significantly attenuated Reg-induced tachycardia. Reg-induced coronary vasodilation was reduced by metoprolol, but the percentage decrease was less than that for HR. These results suggest that regadenoson may be used with a β₁-adrenergic receptor antagonist in MDCT, for the diagnosis of coronary diseases.

### Example 2

Effects of caffeine (1 to 10 mg/kg) on coronary vasodilation and changes in hemodynamics by regadenoson (5 μg/kg, IV) were determined in conscious dogs. Caffeine dose-dependently attenuated the duration of coronary vasodilation, but not the peak increase in coronary hyperemia induced by regadenoson. Caffeine (4 and 10 mg/kg) significantly reduced the effects of regadenoson on mean arterial pressure and heart rate. The results suggest that caffeine consumption immediately prior to pharmacologic stress testing with an A_{2A} adenosine receptor agonist may abbreviate the duration of coronary vasodilation caused by the drug.

### Methods

Sixteen chronically instrumented male mongrel dogs weighing from 22-30 kg were used in the study. The animal protocol was approved by the Institutional Animal Care and Use Committee of New York Medical College and conforms to the Guide for the Care and Use of Laboratory Animal by the United States National Institutes of Health.

Surgical Procedures

Dogs were sedated with acepromazine (0.3 mg/kg, IM) and anesthetized with pentobarbital sodium (25 mg/kg, IV). After intubation, dogs were artificially ventilated with room air. A thoracotomy was made in the fifth intercostal space using sterile techniques. A Tygon catheter (Cardiovascular Instruments, Wakefield, Mass.) was inserted into the descending thoracic aorta and another one was inserted into the left atrium. In 9 dogs, an ultrasound flow transducer (Transonic Systems, Ithaca, N.Y.) was placed around the left circumflex coronary artery. A solid-state pressure gauge (P6.5, Konisberg Instruments, Pasadena, Calif.) was placed into the left ventricle through the apex. The chest was closed in layers. The catheters and wires were tunneled subcutaneously and externalized through the skin at the back of the dog's neck. Dogs were allowed to recover from the surgery before experiments were performed, and were trained to lie on a table.

Coronary Blood Flow and Hemodynamic Measurements

Phasic arterial pressure was measured by connecting the aortic catheter to a strain gauge transducer (P23 ID, LDS Test and Measurement, Valley View, Ohio). Left ventricular pressures were measured by the solid pressure gauge. CBF (mL/min) was measured from an ultrasound flow transducer using a Transonic flowmeter (T206, Transonic Systems, Ithaca, N.Y.). Two indices were used to describe the regadenoson-induced coronary vasodilation: 1) the maximum increase in CBF and 2) the duration of the 2-fold increase in CBF (the period of time that CBF was elevated to a level ≧2-fold of baseline CBF). All pressure and flow data were acquired and analyzed using a Ponemah System (Version 3.30 or 4.20, LDS Test and Measurement, Valley View, Ohio). MAP and HR were calculated from phasic blood pressure, and LV dP/dt_{Max} was calculated from the left ventricular systolic pressure.

Experimental Protocols

On the day of an experiment, a dog was placed on a table, where it lay quietly during the experiment. A catheter was inserted into a peripheral vein in the leg and attached to an infusion line to administer drugs without disturbing the dog. The experiment was begun after MAP, HR and CBF were stable.

### Effects of Caffeine Alone on MAP and HR, and Determination of Plasma Caffeine Concentrations (Part I):

Three experiments were performed on each dog in the group. In each experiment, a dog received an IV injection (over 1 to 3 min) of caffeine at a dose of 2, 4 or 10 mg/kg. Each dog received up to 3 doses of caffeine (on different days) in a random manner. MAP and HR were recorded continuously for 120 min and 3 mL of blood was taken from the aortic catheter at 2.5, 5, 15, 30, 60, 90 and 120 min following administration of caffeine, for measurements of plasma caffeine concentrations.

### Effects of Caffeine on Regadenoson-Induced Coronary Vasodilation and Changes in Hemodynamics (Part II):

Each dog received an IV injection of 5 μg/kg of regadenoson. Forty-five min later, 1 mg/kg of caffeine (IV) was administered. About 45 min after the injection of caffeine, a second-injection of regadenoson was given. LVSP, LV dP/dt_{Max}, MAP, HR and CBF were recorded continuously. Blood samples were taken from the left atrial catheter at 1, 3, 5, 15, 30, 45 and 60 min following injections of regadenoson.

On subsequent days, the protocol and blood sampling were repeated in the same dogs with different doses of caffeine (2, 4 or 10 mg/kg).

In 4 dogs, two doses of regadenoson (5 μg/kg, IV) were given 90 min apart (without blood sampling) to determine if there is tachyphylaxis of regadenoson-induced coronary vasodilation.

Drugs

Regadenoson was supplied by CV Therapeutics, Inc. as a sterile stock solution (Lot#: 803604, 0.08 mg/mL), that was made using 15% Propylene Glycol (pH 7) and was diluted in normal saline before injection. Caffeine was purchased from Sigma-Aldrich (St. Louis, Mo.), and was dissolved in normal saline (10 mg/mL).

Statistical Analysis

The statistical significance of a difference between the value of a parameter at baseline and at the indicated time point after drug administration was determined using a One-Way Repeated Measures ANOVA followed by Tukey's Test. The statistical significance of a difference between responses to regadenoson in the absence and presence of caffeine was determined using a Two-Way Repeated Measures ANOVA followed by Tukey's Test. Results with p<0.05 were considered to be significant. A computer-based software package (SigmaStat 2.03) was used for statistical analysis. All data are presented as Mean±SEM.

### Results

Effects of Caffeine Alone on MAP and HR, and Plasma Caffeine Concentrations

An IV injection of caffeine at 2 mg/kg caused no significant changes in MAP and HR. Caffeine at 4 mg/kg caused a significant increase in MAP by ˜12 mm Hg at both 2.5 and 5 min after injection, without a significant change in HR. Caffeine at 10 mg/kg caused an insignificant increase in MAP (5 to 9 mm Hg at 2.5, 5 and 15 min, p>0.05), but did decrease HR by 16 to 24 beats/min from 30 to 120 min after injection. Plasma caffeine concentrations remained within a relatively narrow range from 30 to 120 min following a caffeine injection (Table 1). Based on these results, it was concluded that 45 min after caffeine administration was optimal for determining the effects of caffeine on regadenoson-induced changes in CBF and hemodynamics.

TABLE 1
Effects of Caffeine (IV) on MAP and HR, and Caffeine Plasma Concentrations in Conscious Dogs

| | Baseline | 2.5 min | 5 min | 15 min | 30 min | 60 min | 90 min | 120 min |
|---|---|---|---|---|---|---|---|---|
| MAP | | | | | | | | |
| (mm Hg) | | | | | | | | |
| 2 mg/kg | 107 ± 4 | 110 ± 5 | 108 ± 3 | 106 ± 4 | 104 ± 4 | 112 ± 5 | 111 ± 7 | 109 ± 6 |
| 4 mg/kg | 97 ± 3 | 109 ± 6* | 108 ± 6* | 99 ± 4 | 103 ± 4 | 104 ± 2 | 108 ± 4* | 104 ± 4 |
| 10 mg/kg | 99 ± 4 | 109 ± 5 | 107 ± 3 | 105 ± 4 | 101 ± 3 | 107 ± 4 | 104 ± 6 | 102 ± 2 |
| HR | | | | | | | | |
| (beats/min) | | | | | | | | |
| 2 mg/kg | 95 ± 6 | 95 ± 5 | 91 ± 5 | 85 ± 6 | 81 ± 7 | 90 ± 9 | 87 ± 5 | 88 ± 6 |
| 4 mg/kg | 100 ± 8 | 104 ± 5 | 102 ± 4 | 88 ± 6 | 90 ± 7 | 85 ± 7* | 90 ± 7 | 86 ± 5 |
| 10 mg/kg | 103 ± 5 | 100 ± 4 | 101 ± 4 | 93 ± 5 | 87 ± 5* | 83 ± 2* | 80 ± 5* | 80 ± 4* |
| Caffeine | | | | | | | | |
| Levels (μM) | | | | | | | | |
| 2 mg/kg | — | 19 ± 0.98 | 15 ± 0.29 | 12 ± 0.19 | 11 ± 0.10 | 9.9 ± 0.11 | 9.1 ± 0.11 | 8.7 ± 0.18 |
| 4 mg/kg | — | 35 ± 0.93 | 28 ± 1.28 | 22 ± 0.89 | 20 ± 0.74 | 17 ± 1.07 | 17 ± 0.64 | 16 ± 0.98 |
| 10 mg/kg | — | 76 ± 3.00 | 67 ± 2.19 | 52 ± 1.37 | 47 ± 2.14 | 45 ± 1.22 | 41 ± 1.78 | 37 ± 1.78 |

MAP: Mean arterial pressure.
HR: Heart rate.
Mean ± SEM, n = 5 (n = 6 for caffeine levels).
Baselines are values before the injection of caffeine.
*p < 0.05, compared with baseline.

### Effects of Caffeine on Regadenoson-Induced Coronary Vasodilation

### Time Control Group:

In 4 dogs, an IV injection of regadenoson (5 μg/kg) caused a significant increase in CBF. The maximum CBF increased from a baseline value of 37±1 to 178±17 mL/min, and the duration of 2-fold increase in CBF was 401±45 sec. A second-injection of regadenoson resulted in an identical coronary vasodilation 90 min later. The maximum CBF increased from a baseline value of 35±1 to 176±6 mL/min, and the duration of 2-fold increase in CBF was 395±43 sec. There were no statistically significant differences in baseline CBFs, in the maximum CBFs or in the duration of 2-fold increase in CBF caused by the two injections of regadenoson.

### Effects of Caffeine on Regadenoson-Induced Coronary Vasodilation:

In the absence of caffeine, an IV injection of regadenoson (5 μg/kg) increased CBF from a baseline value of 34±2 to a peak of 191±7 mL/min, and the duration of the 2-fold increase in CBF caused by regadenoson was 515±71 sec (n=8).

Baseline values of CBFs were not significantly different before and after caffeine treatment (45 min after 1, 2, 4, and 10 mg/kg administration). In the presence of caffeine at 1, 2, 4 and 10 mg/kg, the maximum increases in CBF caused by regadenoson were not significantly reduced from control (in the absence of caffeine). The maximum increases in CBF induced by regadenoson were changed by only 2±3, −0.7±3, −16±5 and −13±8%, respectively, in the presence of caffeine at 1, 2, 4 and 10 mg/kg (all p>0.05). In contrast, the durations of the 2-fold increase in CBF caused by regadenoson were significantly reduced at all dosages of caffeine tested. Reductions of the duration of 2-fold increase in CBF were 17±4, 48±8, 62±5 and 82±5% from control, respectively, in the presence of caffeine at 1, 2, 4 and 10 mg/kg (all p<0.05). However, the regadenoson-increased CBF still remained at ≧2-fold baseline levels for ≧3 min in the presence of 1, 2 and 4 mg/kg caffeine

### Plasma Concentrations of Regadenoson and Caffeine:

In the absence of caffeine, an IV injection of regadenoson (5 μg/kg) caused a short-lasting increase in the plasma regadenoson concentration, which reached at a peak at ˜1 min and decreased rapidly. Pharmacokinetic profiles of regadenoson were not changed by caffeine at 1, 2, 4 or 10 mg/kg.

Plasma caffeine concentrations were 5±0.2, 10±0.6, 18±0.8 and 52±1.8 μM, respectively, at 45 min following administration of caffeine at 1, 2, 4 and 10 mg/kg and immediately before the second injection of regadeno son. Plasma caffeine concentrations remained at relatively steady levels from the time of pre-injection (Time 0) to 30 min following the second injection of regadenoson.

### Effects of Caffeine on Regadenoson-Induced Changes in Hemodynamics

Table 2 shows the values of MAP and HR at different time points following administration of regadenoson either in the absence or presence of caffeine at 1, 2, 4 and 10 mg/kg (The peak responses are not included). Caffeine at 1, 2, 4 or 10 mg/kg did not alter hemodynamics significantly at 45 min following caffeine administration as shown in Table 2 (the baseline values for control and caffeine at 1, 2, 4 and 10 mg/kg).

TABLE 2
Effects of Caffeine on Regadenoson (5 μg/kg, IV)-Induced Changes in MAP
and HR in Conscious Dogs

| | Baseline | 0.5 min | 1 min | 2 min | 3 min |
|---|---|---|---|---|---|
| MAP (mm Hg) | | | | | |
| Control | 104 ± 3 | 97 ± 2 | 93 ± 3* | 92 ± 4* | 92 ± 3* |
| Caffeine (1 mg/kg) | 109 ± 5 | 105 ± 3 | 100 ± 4 | 102 ± 4 | 101 ± 5 |
| Control | 97 ± 3 | 89 ± 5 | 89 ± 5 | 91 ± 5 | 91 ± 3 |
| Caffeine (2 mg/kg) | 110 ± 6† | 106 ± 7† | 102 ± 7† | 104 ± 7† | 106 ± 5† |
| Control | 110 ± 3 | 107 ± 6 | 95 ± 5* | 99 ± 4* | 98 ± 4* |
| Caffeine (4 mg/kg) | 112 ± 3 | 109 ± 5† | 107 ± 5† | 107 ± 4† | 109 ± 3† |
| Control | 99 ± 3 | 93 ± 3 | 86 ± 4* | 89 ± 4* | 89 ± 4* |
| Caffeine (10 mg/kg) | 106 ± 3 | 116 ± 7† | 115 ± 4† | 112 ± 5† | 111 ± 4† |
| HR (bpm) | | | | | |
| Control | 84 ± 6 | 138 ± 10* | 144 ± 13* | 142 ± 9* | 131 ± 9* |
| Caffeine (1 mg/kg) | 74 ± 5 | 126 ± 7* | 135 ± 9* | 131 ± 12* | 119 ± 9* |
| Control | 83 ± 7 | 160 ± 13* | 145 ± 7* | 150 ± 4* | 137 ± 5* |
| Caffeine (2 mg/kg) | 75 ± 5 | 121 ± 10*† | 125 ± 10*† | 122 ± 5*† | 110 ± 3*† |
| Control | 89 ± 7 | 166 ± 18* | 163 ± 8* | 158 ± 6* | 141 ± 4* |
| Caffeine (4 mg/kg) | 81 ± 9 | 126 ± 12*† | 114 ± 11*† | 106 ± 12*† | 102 ± 7† |
| Control | 76 ± 4 | 149 ± 15* | 144 ± 7* | 148 ± 5* | 135 ± 4* |
| Caffeine (10 mg/kg) | 78 ± 6 | 115 ± 12*† | 102 ± 6*† | 106 ± 11*† | 96 ± 7† |

| | 4 min | 5 min | 10 min | 15 min | 20 min |
|---|---|---|---|---|---|
| MAP (mm Hg) | | | | | |
| Control | 94 ± 3* | 96 ± 3* | 97 ± 4 | 96 ± 4 | 96 ± 3 |
| Caffeine (1 mg/kg) | 105 ± 4 | 104 ± 3 | 104 ± 4 | 106 ± 4† | 102 ± 4 |
| Control | 93 ± 4 | 90 ± 3 | 91 ± 2 | 96 ± 3 | 97 ± 3 |
| Caffeine (2 mg/kg) | 105 ± 7† | 103 ± 6† | 106 ± 5† | 107 ± 7† | 111 ± 8† |
| Control | 100 ± 2 | 100 ± 2 | 100 ± 2 | 101 ± 4 | 102 ± 4 |
| Caffeine (4 mg/kg) | 112 ± 3† | 111 ± 5† | 109 ± 3† | 107 ± 3 | 103 ± 1 |
| Control | 92 ± 4 | 92 ± 4 | 95 ± 4 | 93 ± 6 | 98 ± 5 |
| Caffeine (10 mg/kg) | 112 ± 6† | 111 ± 4† | 110 ± 4† | 113 ± 5† | 111 ± 5† |
| HR (bpm) | | | | | |
| Control | 125 ± 8* | 121 ± 8* | 100 ± 7 | 94 ± 7 | 89 ± 7 |
| Caffeine (1 mg/kg) | 110 ± 4*† | 106 ± 7*† | 89 ± 7 | 87 ± 7 | 81 ± 8 |
| Control | 127 ± 4* | 129 ± 6* | 104 ± 5 | 104 ± 6 | 93 ± 7 |
| Caffeine (2 mg/kg) | 106 ± 4*† | 97 ± 3† | 84 ± 5† | 85 ± 6† | 84 ± 5 |
| Control | 131 ± 6* | 128 ± 7* | 113 ± 5 | 102 ± 6 | 101 ± 6 |
| Caffeine (4 mg/kg) | 94 ± 8† | 94 ± 7† | 85 ± 8† | 85 ± 8 | 87 ± 7† |
| Control | 130 ± 5* | 127 ± 6* | 105 ± 4 | 98 ± 3 | 99 ± 7 |
| Caffeine (10 mg/kg) | 94 ± 8† | 93 ± 5† | 88 ± 7† | 88 ± 6 | 86 ± 4 |

MAP: Mean arterial pressure.
HR: Heart rate.
Mean ± SEM, n = 6 (Caffeine 1 mg/kg n = 7, Caffeine 2 mg/kg: n = 5 for MAP).
Baselines are values before the injection of regadenoson. The baselines for caffeine at 1, 2, 4 and 10 mg/kg were the values at 45 min after injection of caffeine.
*p < 0.05, compared with baseline.
†p < 0.05, compared with control.
Note:
In the presence of 2 mg/kg caffeine, values of MAP at all time points were significantly higher than control, however, the delta changes in MAP following IV injection of regadenoson were not statistically different from those in control.

An IV injection of regadenoson (5 μg/kg) caused a mild decrease in MAP. Regadenoson decreased MAP (peak) by 15±2% from a baseline value of 102±2 mm Hg in the absence of caffeine (n=9). In the presence of caffeine at 1 and 2 mg/kg, the peak decrease in MAP caused by regadenoson was unchanged (13±2% vs. 13±1% from baseline, respectively). However, in the presence of 4 mg/kg caffeine, regadenoson decreased peak MAP by only 2±5% from baseline. In the presence of 10 mg/kg caffeine, regadenoson increased MAP, but insignificantly, by 9±6% from baseline.

An IV injection of regadenoson (5 μg/kg) caused an increase in HR lasting for 8 to 9 min. Regadenoson increased HR (peak) by 114±14% from a baseline value of 80±4 beats/min (n=9). Caffeine at 1 mg/kg did not markedly alter the regadenoson-induced tachycardia. Peak HR increased by 124±12% from baseline. Caffeine at 2, 4 or 10 mg/kg significantly attenuated the regadenoson-induced tachycardia in a dose-dependent manner. Peak HRs increased by 109±21%, 79±20%, and 74±16% from baseline, respectively (all p<0.05, compared to control).

Regadenoson decreased LVSP (peak) by 9±1% from a baseline value of 139±5 mm Hg (n=8). In the presence of caffeine at 1 and 2 mg/kg, regadenoson still significantly decreased LVSP by 9±3% and 6±2% from baseline, respectively. In the presence of 4 mg/kg of caffeine, regadenoson caused no a significant decrease in LVSP (1±5% decrease from control, p>0.05), while in the presence of 10 mg/kg caffeine, regadenoson significantly increased LVSP (11±7% increase from control).

An IV injection of 5 μg/kg of regadenoson caused an increase in LV dP/dt_{Max}. Regadenoson increased LV dP/dt_{Max} (peak) by 65±7% from a baseline value of 3240±196 mm Hg/sec. The effects of caffeine on the regadenoson-induced increase in LV dP/dt_{Max} were inconsistent. The increase in LV dP/dt_{Max} caused by regadenoson was slightly greater in the presence of caffeine at 1 mg/kg. In the presence of caffeine at 2 and 4 mg/kg, the regadenoson-induced increase in LV dP/dt_{Max} was slightly smaller. The regadenoson-induced increase in LV dP/dt_{Max} was not altered in the presence of 10 mg/kg caffeine.

Both the magnitude of increase in CBF and the duration of coronary vasodilation are important for accurate diagnosis in myocardial perfusion imaging. The most important finding of the study is that caffeine attenuates the duration of coronary vasodilation, but not the peak increase in CBF in response to regadenoson. Thus, the duration of an A_{2A} receptor-mediated coronary vasodilation is more sensitive than peak CBF to antagonism by caffeine.

Caffeine is a non-specific and unselective antagonist of all adenosine receptor subtypes. The affinities (Ki) of caffeine for human adenosine A₁, A_{2A}, A_{2B} and A₃ receptors are 12, 2.4, 13 and 80 μM, respectively (Fredholm et al. (1999). Pharmacol Rev, 51:83-133). A number of studies have shown that caffeine can attenuate coronary vasodilation induced by adenosine (Smits et al. (1990) Clin Pharmacol Ther, 48:410-8; Kubo et al. (2004) J Nucl Med, 45:730-8; Lapeyre et al. (2004) J Nucl Cardiol, 11:506-11), by dipyridamole (Smits et al. (1991) J Nucl Med, 32:1538-41; Kubo et al. (2004) J Nucl Med, 45:730-8; Lapeyre et al. (2004) J Nucl Cardiol, 11:506-11) and by an A_{2A} receptor agonist, ATL-146e (Riou et al. (2002) J Am Coll Cardiol, 40:1687-94) in humans and dogs. Thus, the action of caffeine can result in false-negative myocardial perfusion imaging in studies using these stress agents (Smits et al. (1991) J Nucl Med, 32:1538-41). However, one report indicated that caffeine did not alter adenosine-induced coronary hyperemia measured by fractional flow reserve in patients with coronary artery disease (Aqel et al. (2004) Am J Cardiol, 93:343-6).

The present results reveal for the first time that caffeine attenuates the regadenoson-induced coronary hyperemia in a unique pattern: caffeine selectively attenuates the duration of regadenoson-induced coronary vasodilation in a dose-dependent manner, but does not markedly alter the maximum increase in CBF. Caffeine at doses of 1 to 10 mg/kg did not reduce the peak plasma regadenoson concentrations, or change the pharmacokinetic profile of regadenoson. The differing affinities of A_{2A} receptor and pharmacokinetic profiles of regadenoson and caffeine might explain the unique pattern of attenuation of coronary hyperemia caused by regadenoson in the presence of caffeine. Immediately after injection, regadenoson molecules could bind most of the A_{2A} receptors in the coronary circulation, thereby causing a similar maximum increase in CBF in the presence of all doses of caffeine. Shortly after injection, plasma regadenoson concentrations decreased rapidly but plasma caffeine concentrations remained relatively constant. Therefore, as caffeine molecules occupy more A_{2A} receptors, the increase in CBF after the peak response to regadenoson would decrease more rapidly in the presence of caffeine, thereby shortening the duration of coronary vasodilation caused by regadenoson. Although these results show that caffeine caused a dose-dependent attenuation of the duration of regadenoson-induced coronary vasodilation in conscious dogs, the regadenoson-increased CBF remained at ≧2-fold of baseline levels for ≧3 min in the presence of caffeine at 1, 2 and 4 mg/kg (equivalent to consumption of 1 to 2 cups of coffee). More recently, it has been reported that one 8-oz cup of coffee taken 1 h prior to adenosine administration did not mask the presence or severity of a reversible defect studied by single-photon emission computed tomography (Zoghbi et al. (2006) J Am Coll Cordiol, 47:2296-302).

Desensitization of the A_{2A} receptor has been reported in cell-based experimental models (Anand-Srivastava et al. (1989) Mol Cell Endocrinol, 62:273-9, Ramkumar et al. (1991) Mol Pharmacol, 40:639-47). However, a related study demonstrated that three successive doses of 1.0 μg/kg regadenoson (5 to 10 min apart) caused similar peak increases in CBF in conscious dogs (Trochu et al. (2003) J Cardiovasc Pharmacol, 41:132-9). Furthermore, in the present study, time control experiments were performed on four conscious dogs to determine if there is tachyphylaxis of the regadenoson-induced coronary vasodilation. The results showed that there were no significant differences either in the maximum increases in CBF or in the duration of 2-fold increase in CBF induced by two injections of regadenoson. Thus, the attenuated coronary hyperemia induced by regadenoson in the presence of caffeine is most likely due to the competitive antagonism of A_{2A} receptors by caffeine.

The present study also showed that IV injection of regadenoson caused mild decreases in MAP (Table 2) and LVSP, and modest increases in HR (Table 2) and LV dP/dt_{Mac} in conscious dogs. The regadenoson-induced changes in MAP and HR in the present study were consistent with related studies. (Trochu et al. (2003) J Cardiovasc Pharmacol, 41:132-9, Zhao et al. (2003) J Pharmacol Exp Ther, 307:182-9) which have indicated that the mild decrease in MAP induced by regadenoson is due to dilation of peripheral vessels. This was evidenced by the reduction of total peripheral resistance (TPR) and dilation of vessels in the lower body by regadenoson (Zhao et al. (2003) J Pharmacol Exp Ther, 307:182-9).

Caffeine has been shown to attenuate the dipyridamole-induced increase in blood pressure in humans in a dose-dependent manner (Smits et al. (1991) Clin Pharmacol Ther, 50:529-37). The present study further confirmed that caffeine caused a dose-dependently attenuation of hypotension induced by regadenoson, a novel adenosine A_{2A} receptor agonist, in conscious dogs It was reported that adenosine could increase sympathetic nerve activity in humans, thereby causing tachycardia (Biaggioni et al. (1991) Circulation, 83:1668-75). The present results showed that an IV injection of regadenoson caused a significant tachycardia in conscious dogs, and are consistent with related studies (Trochu et al. (2003) J Cardiovasc Pharmacol, 41:132-9, Zhao et al. (2003) J Pharmacol Exp Ther, 307:182-9). More importantly, one recent study indicated that the regadenoson-induced tachycardia in awake rats is directly mediated by sympathoexcitation (Dhalla et al. (2006) J Pharmacol Exp Ther, 316:695-702), in which the regadenoson-induced tachycardia was abolished by hexamethonium (a ganglionic blocker). The present study demonstrated that caffeine attenuated regadenoson-induced tachycardia in a dose-dependent manner in conscious dogs. However, the mechanism(s) for the reduction by caffeine of tachycardia induced by regadenoson remains to be determined.

In summary the result of the example above indicate that doses of 1 to 10 mg/kg IV caffeine:
(1) did not alter baseline CBF and hemodynamics at 45 min, when caffeine plasma concentrations were as high as 52±2 μM;
(2) did not significantly reduce the regadenoson-induced peak increases in CBF;
(3) caused a dose-dependent decrease in the duration of the regadenoson-induced coronary vasodilation; and
(4) blunted the regadenoson-induced sinus tachycardia and hypotension.

### Example 3

### Objectives

The primary objective was to evaluate the effect of a 200-mg oral dose of caffeine on the regadenoson-induced increase in myocardial blood flow (MBF), measured approximately 2 hours after caffeine ingestion. Secondary objectives included the following:
To evaluate the regadenoson-induced heart rate (HR) response with and without prior caffeine;
To evaluate the relationship between the regadenoson-induced increase in MBF and HR changes, and whether it is altered by oral caffeine;
To evaluate the regadenoson-induced blood pressure (BP) response with and without prior caffeine;
To assess the safety and tolerability of regadenoson with and without prior caffeine; and
To assess whether the effect of prior caffeine on the MBF response to regadenoson differs between male and female volunteers.

### Methodology:

This was a randomized, double-blind, crossover study of regadenoson in normal subjects with and without caffeine. Resting and stress positron emission tomography (PET) scans were performed following regadenoson administration (a single 400 μg intravenous (IV) dose, administered over 10 seconds, followed by a 5 mL saline flush) and following dosing with caffeine 200 mg or placebo on each of 2 study days. 15O water was used as the radionuclide in the PET scans. There was a 1- to 14-day washout period between dosing days. Blood samples and measures of safety were collected until 120 minutes after study drug administration.

### Number of Subjects (Planned and Analyzed):

The study was designed to enroll 52 subjects (26 in each crossover sequence) in order that 40 subjects complete the study with evaluable data. There were 45 subjects enrolled and randomized and 43 subjects dosed with regadenoson of which 41 subjects completed the study, 40 subjects were evaluable for efficacy, and 2 subjects terminated prematurely.

### Diagnosis and Main Criteria for Inclusion:

Healthy adult men or women (≧18 years of age) who provided written informed consent, and who were nonsmokers and regular coffee drinkers (at least one cup per day) were considered for inclusion in the study. Enrolled subjects were to have had no clinically relevant physical findings or electrocardiogram (ECG) findings at baseline. They were also required to abstain from intake of caffeine or other methylxanthines for 24 hours before each study day, and to abstain from all food and beverages except water from 4 hours before the baseline assessments until the final blood sample was taken (5 minutes after the stress PET scan). Female subjects of childbearing potential must have had a negative baseline pregnancy test and have used an acceptable method of birth control for 3 months prior to admission and through 1 week following the study.

Subjects were not eligible for enrollment in the study if they had any illness requiring ongoing treatment. Those with a history of alcohol abuse or drug addiction, or a history of known or suspected bronchoconstrictive and bronchospastic lung disease, or a known allergy to theophylline or aminophylline were not permitted to enroll.

### Test Product, Dose and Mode of Administration, Batch Number:

Open-label study drug was supplied as sterile stock solution in single-use vials each containing 5 mL of regadenoson (0.08 mg/mL). Regadenoson, 400 μg, was administered as a rapid bolus, through an IV catheter over approximately 10 seconds, followed immediately by a 5 mL saline flush. Regadenoson (study drug) had the following CVT lot number: 803604.

### Duration of Treatment:

On each of 2 study days, subjects received a single dose of regadenoson, administered intravenously as a rapid (10-second) bolus of 5 mL, followed by a 5 mL saline flush. There was a 1- to 14-day washout period between doses.

### Reference Therapy, Dose and Mode of Administration, Batch Number:

Caffeine, 200 mg po, or placebo capsule was administered approximately 105 minutes prior to regadenoson. The CVT tracking number for the caffeine capsules was 1341 (Leg 3). These capsules contained caffeine tablets from Bristol-Myers Squibb (NoDoz®) with lot number 405542. The CVT tracking number for the placebo capsules was 1341 (Leg 2).

### Criteria for Evaluation:

### Efficacy:

The primary efficacy measure was the log coronary flow reserve (CFR), which is the ratio of stress MBF after regadenoson dosing to the resting MBF. Plasma caffeine, theophylline, and regadenoson concentrations were measured, and were to be used in exploratory analyses.

### Safety:

Safety measures included adverse events (AEs), serious adverse events, vital signs (HR and BP), ECG, concomitant medications, and a tolerability questionnaire. All available data from subjects who received the single dose of regadenoson were to be included in the statistical summaries.

The primary efficacy analysis was to test whether caffeine reduces CFR after regadenoson administration by at least 10%, using an analysis of variance (ANOVA) with terms for sequence, subject-within-sequence, period, and treatment. The limits of the 95% and 90% confidence intervals (CIs) for the difference of treatment mean values (caffeine-placebo; log scale) were to be exponentiated to obtain CIs for the ratios of the raw scale median values. If the lower limit of this latter 90% CI exceeded 0.9, it could be stated with 95% confidence that prior caffeine administration reduces CFR by less than 10%. The data were also to be analyzed using Wilcoxon's rank-sum test.

The effect of caffeine was to be compared in male and female subjects. Exploratory pharmacodynamic analyses included effect of caffeine on HR and BP and on the relationship between MBF and HR/BP, as well as the correlation between CFR and plasma caffeine concentrations. AEs occurring or worsening after regadenoson administration were to be summarized by severity, relationship to study drug, and prior caffeine status. Vital signs (HR, systolic and diastolic BP, and calculated mean arterial pressure) were to be summarized at individual time points and change-from-baseline values were to be calculated; CIs for the difference in mean values (caffeine-placebo) were to be determined.

Relationships between caffeine and theophylline plasma concentrations and HR and BP were to be explored. ECG intervals and changes from baseline values in ECG intervals were to be presented, as were occurrences of rhythm or conduction abnormalities. Concomitant medication usage was to be summarized.

Tolerability questionnaire responses were to be analyzed using the Wilcoxen rank sum test (“How did you feel?” question) and the exact Cochran-Mantel-Haenszel test (Day 2-only question “How did this test compare to the first test?”).

### Efficacy Results:

The log CFR±SE for the placebo group (n=40) was 1.03±0.06 and log CFR for the caffeine group (n=40) was 0.95±0.06. The CFR (stress/rest) for the placebo group was 2.97±0.16 and for the caffeine group was 2.75±0.16.

While there was no change in CFR detected in this study, the study does not rule out nor does it establish a significant interaction between regadenoson and caffeine on log CFR. The exponentiated upper and lower limits of the 95 and 90% confidence intervals for log CFR (caffeine versus placebo difference) are 1.08 and 0.78 and 1.06 and 0.80, respectively.

Since this lower limit is less than 0.9, but the upper limit is >1, this study cannot establish or rule out an interaction. However, there is 95% confidence that the change in CFR is not ≧20%.

There was no significant interaction of caffeine with regadenoson on CFR by sex.

### Safety Results:

AEs occurred at any time in the following classes by percentage of subjects: cardiac disorders 25/43 (58%), respiratory, thoracic and mediastinal disorders 25/43 (58%), nervous system disorders 18/43 (42%), vascular disorders 13/43 (30%), musculoskeletal and connective tissue disorders 12/43 (28%), general disorders and administration site conditions 11/43 (26%), gastrointestinal disorders 2/43 (5%), and ear and labyrinth disorders 1/43 (2%).

The most frequently occurring AEs were dyspnoea 24/43 (56%), palpitations 21/43 (49%), flushing 13/43 (30%), headache 12/43 (28%), sensation of heaviness 12/27 (28%), and paraesthesia 8/43 (19%).

Forty percent (17/43) of subjects had at least one AE with a maximum severity of mild, 49% (21/43) moderate, and 9% (4/43) severe. Ninety-five percent of subjects (41/43) had at least one AE that was considered probably related and 2% (1/43) of patients had at least one AE that was considered possibly related to regadenoson treatment.

Regadenoson-induced headache severity was decreased with caffeine (p=0.012). There were no reported deaths or SAEs.

Caffeine attenuated the HR increase caused by regadenoson (p<0.001). There was no effect of caffeine on systolic or diastolic blood pressures in the presence of regadenoson.

After regadenoson dosing, one subject appears to have developed first degree AV block, and one subject appears to have had QTc prolongation (>500 msec and change of >60 msec) as determined by ECG analysis that were not reported as AEs.

According to the tolerability questionnaire, subjects felt more comfortable during the test with caffeine (p<0.001), and felt better after the caffeine test than after the placebo test (p<0.001).

While there was no change in CFR detected in this study, the study does not rule out nor does it establish a significant interaction between regadenoson and caffeine on log CFR. The exponentiated upper and lower limits of the 95 and 90% confidence intervals for log CFR (caffeine versus placebo difference) are 1.08 and 0.78 and 1.06 and 0.80, respectively.

Since this lower limit is less than 0.9, but the upper limit is >1, this study cannot establish or rule out an interaction. However, there is 95% confidence that the change in CFR is not ≧20%.

There was no significant interaction of caffeine with regadenoson on CFR by sex.

There was no difference in overall incidence of AEs between the placebo and caffeine groups; however, caffeine attenuated the severity of AEs. Regadenoson-induced headache severity was decreased with caffeine.

1. A pharmaceutical composition comprising a rate control agent, at least 10 μg of at least one A_{2A} receptor agonist, and at least one pharmaceutically acceptable carrier.

2. The pharmaceutical composition of claim 1, wherein the rate control agent is a non-selective adenosine antagonist.

3. The pharmaceutical composition of claim 1, wherein the rate control agent is selected from the group consisting of caffeine, aminophylline caffeine, dyphylline, enprophylline, pentoxyphylline, theophylline, a β-adrenergic receptor blocker, and combinations thereof.

4. The pharmaceutical composition of claim 3, wherein the β-adrenergic receptor blocker is selected from the group consisting of acebutolol, albuterol, amosulalol, arotinolol, atenolol, befunolol, betaxolol, bevantolol, bisoprolol, bisoprolol fumarate, bopindolol, bucindolol, bufetolol, bunitrolol, butaxamine, butofilolol, carazolol, carteolol, carvedilol, celiprolol, cloranolol, divalproex, epanolol, carvedilol, esmolol, indenolol, landiolol, labetalol, levobunolol, levomoprolol, lisinopril, medroxalol, mepindolol, metipranolol, metoprolol, nadolol, nebivolol, nifenalol, nipradilol, oxprenolol, penbutolol, pindolol, propafenone, propranolol, salmeterol, sotalol, talinolol, tertatolol, tilisolol, timolol, verapamil, xamoterol, xibenolol, and combinations thereof.

5. The pharmaceutical composition of claim 1, wherein the A_{2A} receptor agonist is selected from the group consisting of regadenoson, binodenoson, CVT-3033, and combinations thereof.

6. The pharmaceutical composition of claim 1, wherein the A_{2A} receptor agonist is regadenoson.

7. The pharmaceutical composition of claim 1, wherein the A_{2A} receptor agonist is regadenoson and the rate control agent is selected from the group consisting of caffeine, aminophylline caffeine, dyphylline, enprophylline, pentoxyphylline, and theophylline, metoprolol, and propranolol.

8. A method of vasodilator induced myocardial stress perfusion multidetector computed tomography imaging of a myocardium of a mammal, comprising administering a therapeutically effective amount of a rate control agent and at least 10 μg of at least one A_{2A} receptor agonist to the mammal and imaging the myocardium of the mammal.

9. A method of vasodilator induced myocardial stress perfusion multidetector computed tomography imaging of a myocardium of a mammal, comprising administering a therapeutically effective amount of a rate control agent and no more than about 1000 μg of at least one A_{2A} receptor agonist to the mammal and imaging the myocardium of the mammal.

10. The method of claim 8 or 9, wherein the rate control agent is administered to the mammal before or concurrently with the at least one A_{2A} receptor agonist.

11. The method of claim 10, wherein A_{2A} receptor agonist is administered in an amount ranging from about 10 to about 600 μg to the mammal.

12. The method of claim 8 or 9, wherein the A_{2A} receptor agonist is administered in less than about 10 seconds.

13. The method of claim 8 or 9, wherein the A_{2A} receptor agonist is administered in an amount greater than about 10 μg.

14. The method of claim 8 or 9, wherein the A_{2A} receptor agonist is administered in an amount greater than about 100 μg.

15. The method of claim 8 or 9, wherein the A_{2A} receptor agonist is administered in an amount no greater than 600 μg.

16. The method of claim 15, wherein the A_{2A} receptor agonist is administered in an amount no greater than 500 μg.

17. The method of claim 8 or 9, wherein the A_{2A} receptor agonist is administered in an amount ranging from about 100 μg to about 500 μg.

18. The method of claim 8 or 9, wherein the A_{2A} receptor agonist is selected from the group consisting of CVT-3033, regadenoson, and combinations thereof.

19. The method of claim 8 or 9, wherein the rate control agent is selected from the group consisting of caffeine, aminophylline caffeine, dyphylline, enprophylline, pentoxyphylline, theophylline, β-adrenergic receptor blockers, and combinations thereof.

20. The method of claim 19, wherein the β-adrenergic blocker is selected from the group consisting of acebutolol, albuterol, amosulalol, arotinolol, atenolol, befunolol, betaxolol, bevantolol, bisoprolol, bisoprolol fumarate, bopindolol, bucindolol, bufetolol, bunitrolol, butaxamine, butofilolol, carazolol, carteolol, carvedilol, celiprolol, cloranolol, divalproex, epanolol, carvedilol, esmolol, indenolol, landiolol, labetalol, levobunolol, levomoprolol, lisinopril, medroxalol, mepindolol, metipranolol, metoprolol, nadolol, nebivolol, nifenalol, nipradilol, oxprenolol, penbutolol, pindolol, propafenone, propranolol, salmeterol, sotalol, talinolol, tertatolol, tilisolol, timolol, verapamil, xamoterol, xibenolol, and combinations thereof.

21. The method of claim 20, wherein the β-adrenergic blocker is selected from metoprolol or propranolol.

22. The method of claim 8 or 9, wherein the mammal is a human.

23. The method of claim 8 or 9, wherein the A_{2A} receptor agonist is administered in a single IV bolus.
